(19) United States
(12) Patent Application Publication
(10) Pub. No.: US 2023/0105701 A1
(43) Pub. Date: Apr. 6, 2023

(54) DISSOLUTION-ENHANCED OLAPARIB COMPOSITION

(71) Applicant: SHANGHAI INSTITUTE OF MATERIA MEDICA, CHINESE ACADEMY OF SCIENCE, Shanghai (CN)

(72) Inventors: Yong GAN, Shanghai (CN); Shiyan GUO, Shanghai (CN); Wei AN, Shanghai (CN)

(21) Appl. No.: 17/908,990

(22) PCT Filed: Mar. 4, 2021
(86) PCT No.: PCT/CN2021/078978

§ 371 Date: Sep. 2, 2022

(30) Foreign Application Priority Data
Mar. 4, 2020 (CN) 202010143408.X

(51) Int. Cl. A61K 9/20; A61K 31/502

(52) U.S. Cl. CPC A61K 9/205; A61K 31/502; A61K 9/2027

(57) ABSTRACT

Provided is a dissolution-enhanced olaparib composition, a preparation method therefor, a use thereof, and a medicament including the dissolution-enhanced olaparib composition. The dissolution-enhanced olaparib composition includes: olaparib; copovidone and a dissolution enhancer, wherein based on 100 parts by weight of olaparib, 100 or more and less than 200 parts by weight of copovidone, and 20 to 150 parts by weight of a dissolution enhancer. The dissolution-enhanced olaparib composition and the medicament prepared therefrom have controllable stability, increased oral absorption of the active ingredient, reduced excipient dosage, improved medication convenience, and are easy for industrial production.

### TECHNICAL FIELD

The present invention relates to the technical field of olaparib preparations, and in particular, to a dissolution-enhanced olaparib composition, a preparation method thereof, a use thereof for preparing a medicament for preventing or treating a tumor, and a medicament comprising the dissolution-enhanced olaparib composition.

### BACKGROUND ART

As WHO interprets tumors as a controllable and curable chronic disease, the notion of treatment of tumors in recent years has shifted from traditional “tumor-free survival” to “tumor-bearing survival”, and from over-treatment and pure pursuit of survival rate in the past to both survival and life quality. The “tumor-bearing survival” remarkably prolongs the survival period of the patient compared with the traditional chemotherapy (about 5-10 years VS about 1 year), and this chronic treatment mode promotes the change in the medication mode of the patient from injection to oral administration that is convenient for long-term use, and in the treatment mode from the traditional chemotherapy with serious toxic and side effects to high-efficiency and low-toxicity molecular targeted therapy, opening a new epoch of tumor treatment.

Ordinary molecular targeted therapy has no differentiating medication for patients. Although it has significantly reduced side effects compared with traditional chemotherapy, its clinical application is limited by dose-limiting toxicity and some special adverse reactions. Individualized precision therapy accurately uses targeted therapy drugs based on the patient's genetic changes, individual characteristics, etc. to achieve lower toxicity and better efficacy, and is the future direction of tumor treatment. Among them, PARP inhibitors selectively kill tumor cells by using defects in DNA repair pathways, to which the 2015 Nobel Prize in Chemistry was awarded, and they are typical representatives of new anti-tumor precision treatment drugs developed by major international pharmaceutical companies in the past 10 years. PARP inhibitors have therapeutic potential for a wide range of tumors with DNA repair defects, can be used singly for tumors such as breast cancer and ovarian cancer, and can also be used as a basic therapy drug in combination with various chemotherapeutic drugs and tumor immunotherapy drugs.

Olaparib has a chemical name of 1-(cyclopropanoyl)-4-[5-[(3,4-dihydro-4-oxo-1-phthalazinyl)methyl]-2-fluorobenzoyl]piperazin e with a molecular formula of C₂₄H₂₃FN₄O₃ and a molecular weight of 434.46. The US Food and Drug Administration (FDA) approved its capsules in December 2014 and its tablets in August 2017. It is the first PARP inhibitor approved for marketing (subsequently approved by EMA, Japan, China, etc.). Among the 4 PARP inhibitors (Olaparib, Lucaparib, Liraparib, and Tarazoparib) that have been marketed in the world, Olaparib has the advantages of the widest indications, the smallest off-target effect, the lowest adverse reactions and the greatest market potential. So far, only olaparib has been approved by the FDA and CDE and recommended by NCCN guidelines for the first-line maintenance treatment of ovarian cancer. In 2018, PARP inhibitors had a market scale close to US$ 1 billion, and olaparib accounted for more than half of the market share (US$ 647 millions)), and its good curative effect has been clinically confirmed.

Olaparib has a solubility of less than 0.2 mg/ml within the physiological pH range and a medium permeability, and belongs to the BCS IV drug (CN102238945B, US20170105937A1). The ordinary preparations of olaparib, when directly administered, have poor in vivo absorption and low bioavailability, and cannot effectively play the curative effects. Therefore, it needs to be used after improving oral absorption by solubilization. The marketed capsules use Glucire 44/14 to solubilize olaparib. However, this excipient has limited solubilization ability, and the drug needs to be micronized. The process is complicated and the drug in the capsule is in a suspended state. The oral absorption of the drug is only improved to a limited extent (the bioavailability is about 10%-20%) even a large amount of excipient is used. The patient needs to take 16 0 # capsules (the daily dose is 800 mg, 50 mg/capsule), which is not convenient for medication (CN102238945B). The marketed tablets use copovidone as the matrix polymer (FDA Olaparib tablet Review, Reference ID: 4139600), and hot melt extrusion technology is used to prepare olaparib into a solid dispersion to improve solubilization and absorption, in which the bioavailability of the drug is improved compared with the capsule, and the recommended daily dose is 600 mg, improving the patient's compliance. However, this preparation solely uses copovidone as the matrix, and if mannitol is not added as the excipient to assist the dissolution of the drug, the drug cannot be effectively dissolved. The main specification of the marketed preparation is 150 mg (the drug accounts for 24.2%), and the weight of a single tablet is about 620 mg with a large amount of excipient, and it is difficult for patients with advanced cancer to swallow, which limits the introduction of high-dose preparations.

It can be seen that it is necessary to reduce the amount of excipients while improving the oral absorption of the active ingredient to increase the convenience of patients' medication, and to provide an improved preparation for oral administration of olaparib.

After patent search, the preparation patents related to olaparib solubilization for improvement of oral absorption include: olaparib solid dispersion and its tablets (WO2010041051, CN102238945B), olaparib solid dispersion and its granules, tablets and capsules (CN104434809B), olaparib solid dispersion (EP3263095), olaparib solid dispersion and its granules, powders and capsules (CN106692066A), etc. The details are as follows:

1) WO2010041051 and CN102238945B disclose an olaparib solid dispersion preparation, wherein the olaparib solid dispersion is prepared with copovidone as the main material, the weight ratio of olaparib and copovidone is 1:2 to 1:4, and the active agent accouts for 20% to 30%. Since olaparib is difficult to dissolve after pulverized with copovidone and pressed into tablets, it is necessary to add 14.7% by weight of mannitol so that the drug can be dissolved effectively, and thus the amount of excipients is large, and there is a problem of difficulty in swallowing for patients with advanced cancer, which limits the development of high-dose preparations.

2) CN104434809B discloses a solid dispersion of olaparib, wherein the solid dispersion of olaparib is prepared with povidone as the main material, and the ratio of olaparib to polymer is 25 to 100 parts of olaparib to 50-250 parts of povidone. The preparation of the tablets requires addition of disintegrant and a large amount of diluents, and the amount of excipients is large, and povidone has disadvantages of poor thermal stability and easy degradation and blackening when the solid dispersion is prepared by melt extrusion.

3) EP3263095 discloses an olaparib solid dispersion preparation, wherein the solid dispersion is prepared with a hydrophilic polymer with a glass transition temperature of 40-100° C., and the ratio of olaparib to the polymer is 1:0.5 to 1:5, preferably 1:1 to 1:3. Specifically, the hydrophilic polymer is selected from Soluplus and Eudragit series. The used polymer has low glass transition temperature, and the risk of crystallization of the drug during storage is high. In many examples of EP3263095, after 10 days of storage at 40° C. and 75% RH (Eudragit E100 1:1, 1:3), the DSC curves showed an endothermic peak, indicating a poor stability of the solid dispersion.

4) CN106692066A discloses a preparation method of an olaparib solid dispersion and its products, wherein the solid dispersion is prepared by melt extrusion, the polymer used is povidone K30 and copovidone, the weight percentage of olaparib is 5% to 30%, and the weight percentage of the polymer is 70% to 95%. There are disadvantages that the polymer content ratio is high and povidone is not suitable for high temperature (200° C.) extrusion.

It can be seen from the patent search results that the existing olaparib solid dispersion preparations need to use a large amount of excipients to improve the bioavailability of the drug, and there are problems that the patients have problems in convenience when administering at a large dose and the solid dispersions prepared with small amount of excipients or the polymers with a low glass transition temperature have problems in stability or limited solubilization capability.

### SUMMARY OF THE INVENTION

To meet the needs for improving the existing olaparib solid dispersion preparations, the present invention has developed a dissolution-enhanced olaparib composition, which can increase the oral absorption of the active ingredient and reduce the amount of excipients by synergistically using copovidone and a water-soluble cyclodextrin derivative that is a dissolution enhancer. In addition, the dissolution-enhanced olaparib composition of the present invention is controllable in stability with increased medication convenience for patients. The synergistic use of copovidone and dissolution enhancer in the present invention can significantly improve the dissolution behavior of the active ingredient such that the solubilizing capacity and the dissolution enhancing capacity are higher than those using copovidone singly under the same proportion, and the stability is improved compared with that using single dissolution enhancer singly. The dissolution-enhanced olaparib composition and the medicament prepared therefrom of the present invention have stable dissolution behavior and improved bioavailability of the active ingredient, and easy for industrial production.

In the present invention, the term “solubilization” refers to increasing the amount of a drug present in molecular form (dissolved amount) in a solution (including gastrointestinal digestive juice); and the term “dissolution enhancing” refers to enhancing the ability of a drug to dissolve from a certain preparation form into a certain medium (percent dissolution).

According to the first aspect of the present invention, provided is a dissolution-enhanced olaparib composition, which comprises olaparib; copovidone and a dissolution enhancer.

In the dissolution-enhanced olaparib composition of the present invention, based on 100 parts by weight of olaparib, the copovidone may be 100 or more and less than 200, preferably 150 to 195 parts by weight, and the dissolution enhancer may be 20 to 150, preferably 25 to 120 parts by weight.

The dissolution enhancer has a compound function of increasing the solubility of the active ingredient and promoting the dissolution of the drug from the preparation. Preferably, the dissolution enhancer is selected from water-soluble cyclodextrin derivatives, preferably one or a combination of two or more selected from methyl-β-cyclodextrin, hydroxypropyl-β-cyclodextrin, sulfobutyl-β-cyclodextrin and hydroxypropyl-γ-cyclodextrin, more preferably one selected from hydroxypropyl-β-cyclodextrin and sulfobutyl-β-cyclodextrin or a combination thereof.

The dissolution-enhanced olaparib composition of the present invention may further comprise other pharmaceutical excipients, such as a surfactant, a glidant, a lubricant, a plasticizer, etc.

The surfactant can further enhance the improvement of the therapeutic potential of the composition of the present invention. A suitable surfactant may be one or more selected from sodium lauryl sulfate, docusate sodium, cetrimide, benzethonium chloride, cetylpyridinium chloride, lauric acid, polyoxyethylene alkyl ether, sorbitan fatty acid ester, polyoxyethylene sorbitan fatty acid ester, polyoxyethylene castor oil derivative, polyoxyl 40 stearate, octyl/decyl mono and diglycerides, polyoxyethylene stearate and poloxamer, but not limited thereto. Based on 100 parts by weight of olaparib, the surfactant may be 0 to 20, preferably 0 to 10, for example, 0.1 to 10 parts by weight.

The glidant can solve the fluidity problem of materials in industrial production. A suitable glidant may be one or more selected from colloidal silica, animal or vegetable fats, and waxes, but are not limited thereto. Based on 100 parts by weight of olaparib, the glidant may be 0 to 15, preferably 0 to 10, for example, 0.1 to 10 parts by weight.

The lubricant may be one or more selected from polyethylene glycol (e.g., with a molecular weight of 8000 to 6000), magnesium stearate, calcium stearate, sodium stearyl fumarate, glyceryl mono/di-behenate, polyethylene glycol-8-glyceryl behenate and glyceryl distearate, but not limited thereto. Based on 100 parts by weight of olaparib, the lubricant may be 0 to 15, preferably 0 to 10, for example, 0 to 5 or 0.1 to 5 parts by weight.

The plasticizer can improve the processability of the composition. A suitable plasticizer may be one or more selected from acetyl tributyl citrate, acetyl triethyl citrate, benzyl benzoate, chlorobutanol, dextrin, dibutyl phthalate, diethyl phthalate, dimethyl phthalate, glycerin, glyceryl monostearate, mineral oil, lanolin alcohol, palmitic acid, polyethylene glycol, polyvinyl acetate phthalate, propylene glycol, 2-pyrrolidone, stearic acid, triacetin, tributyl citrate, triethanolamine, and triethyl citrate, but not limited thereto.

The amount of the conventional pharmaceutical excipients such as the plasticizer can be easily determined by those skilled in the art as needed.

In the present invention, the copovidone has the function of increasing the solubility of olaparib. However, the inventors found that olaparib cannot be effectively dissolved after tableting copovidone and olaparib (Comparative example 2, where the dissolution is less than 40% in 3 hours), and a further pharmaceutical excipient for enhancing dissolution such as mannitol must be added so that olaparib can be dissolved smoothly (Comparative example 1, where the dissolution is greater than 80% in 60 minutes). Therefore, the amount of excipients in the preparation of the prior art is relatively large (the commercially available tablet weighs 620 mg for a 150 mg specification), which causes the tablet is too large in size and is not easy to take by patients, particularly limiting the development of a preparation with a large dose. The present inventors surprisingly discovered through experiments that copovidone together with the dissolution enhancer of the present invention can play a synergistic effect, not only can enhance dissolution and increase dissolution stability, but also further increase the solubility of olaparib. Under the same weight ratio of olaparib and excipients, the composition comprising both of copovidone and the dissolution enhancer of the present invention has a stronger solubilizing ability than that using copovidone alone (990 μg/ml vs.780 μg/ml), and an improved dissolution stability than that using the dissolution enhancer alone (stable dissolution vs significantly decreased dissolution after stored for 6 months under accelerated conditions), and can promote the dissolution of olaparib in the pharmaceutical composition prepared therewith without a filler/disintegrant (dissolution greater than 80% in 60 min). In addition, the dissolution-enhanced olaparib composition of the present invention is beneficial to reduce the amount of excipients such as copovidone (compared to the commercially available tablets, the ratio of olaparib to copovidone is reduced from 1:2.3 to 1:2 or less, so that the amount of copovidone is reduced by 10% or more), and the convenience of medication for patients is increased.

The experimental results show that, by using the copovidone and the dissolution enhancer of the present invention, the supersaturated solubility of olaparib in a citrate buffer system at pH 4.0 and 37° C. for 2 hours is greater than 800 μg/ml, which is higher than that using copovidone alone (780 μg/ml) under the same ratio. In particular, in an example of the present invention, the supersaturated solubility of olaparib in a citrate buffer system at pH 4.0 and 37° C. for 2 hours is 990 μg/ml, which is superior to that of the solid dispersion prepared by using copovidone alone as a solubilizer (780 μg/ml) under the same ratio.

In an example of the present invention, comparing with the preparations prepared by compound utilization of copovidone with sodium dodecyl sulfate (SDS), hypromellose (HPMC) E5 or hypromellose acetate succinate (HPMCAS), which are the excipients with solubilization and/or dissolution promotion effects that are commonly used in the art, the combinaton of copovidone and the water-soluble cyclodextrin derivative as the dissolution enhancer of the present invention can further improve the supersaturated solubility of olaparib. Compared with the effects of the combination of povidone and the water-soluble cyclodextrin derivative as the dissolution enhancer on olaparib solubilization, the combination of copovidone and the water-soluble cyclodextrin derivative as the dissolution enhancer of the present invention showed better improvement effects in supersaturated solubility of olaparib (990.1 μg/ml VS 825.5 μg/ml), and a significantly higher in vivo AUC.

According to the second aspect of the present invention, provided is a method for preparing the dissolution-enhanced olaparib composition, which comprises a step of mixing olaparib with copovidone, the dissolution enhancer and the optional other pharmaceutical excipients uniformly to obtain a uniform solid dispersion. In particular, the mixing may be a formulation process as follows, for example, it may be a solvent evaporation method, including rotary evaporation, spray drying, lyophilization and thin-film evaporation; or, the solvent can be removed by cryofreezing followed by lyophilization; or it may use other techniques such as melt extrusion, solvent-controlled precipitation, pH-controlled precipitation, and cryogenic co-milling.

According to the third aspect of the present invention, provided is use of the dissolution-enhanced olaparib composition in preparation of a medicament. In some embodiments, the medicament may be tablets, capsules, granules, pills, powders, etc., but is not limited thereto.

The medicament can be used to prevent or treat a tumor. Preferably, the tumor is selected from tumors with defective DNA repair function, especially cancers associated with two or more BRCA gene mutations, such as ovarian cancer, gastric cancer, breast cancer, etc., and tumors associated with BRCA1 and BRCA2 gene mutations, but not limited thereto.

According to the fourth aspect of the present invention, provided is a dissolution-enhanced olaparib medicament, which comprises the dissolution-enhanced olaparib composition of the present invention. Specifically, the dissolution-enhanced olaparib medicament according to the present invention is prepared by using the dissolution-enhanced olaparib composition of the present invention.

The olaparib medicament provided by the present invention has controllable physical stability without slowing down the dissolution after being placed under accelerated conditions (40° C., 75% RH), showing improved stability than the cases of using the dissolution enhancer alone, combination utilization of povidone and the dissolution enhancer, combination of copovidone and the dissolution enhancer outside the dosage range of the present invention in the same ratio of olaparib to the excipients. In an example of the present invention, the dissolution-enhanced pharmaceutical composition of olaparib, after placed under accelerated conditions (40° C., 75% RH) for 6 months, has a consistent dissolution with that of 0 months, showed a good stability; under the same content of excipients, the preparation prepared by using the dissolution enhancer alone, after placed for 6 months, showed a slowed-down dissolution; under the same content of excipients, the preparation prepared by compound utilization of povidone and the dissolution enhancer, after placed for 6 months, showed a slowed-down dissolution; and under the same content of excipients, the preparation prepared by combination of copovidone and the dissolution enhancer outside the dosage range of the present invention, after placed for 6 months, showed a slowed-down dissolution.

The present inventors surprisingly discovered that, due to the combination of copovidone and the dissolution enhancer, even the dissolution-enhanced olaparib composition of the present invention is prepared into tablets without additional addition of a filler or a disintegrant, olaparib can still be effectively dissolved with good in vivo absorption. Compared with existing preparations, the present invention can reduce the amount of excipients, decrease the size of tablets, which is beneficial for the patient to swallow, and achieve a higher single dose according to the needs of clinical large-dose administration. In an example of the present invention, the dissolution-enhanced olaparib pharmaceutical composition of the present invention does not use a filler and a disintegrant, and has a dissolution behavior similar to that of the marketed preparation (both with a specification of 150 mg) in a pH 4.0 medium, wherein the dissolution in 60 min is greater than 80%, and the tablet weighs 500 mg, which is 120 mg lower than the commercially available tablet that weighs 620 mg, and the tablet weight is greatly reduced (19% reduction), which is beneficial for the patient to swallow. In an example of the present invention, for the dissolution-enhanced olaparib pharmaceutical composition of the present invention, when the tablet specification is 200 mg, the coated tablet weight is 667 mg, which is equivalent to that of the commercially available tablet with a specification of 150 mg; and when the tablet specification is 250 mg, the coated tablet weight is 883 mg, while the tablet weight of the commercially available tablet will be 1033 mg when it was converted to the 250 mg specification. So the pharmaceutical composition of the present invention can meet the needs of the patient for high-dose administration and increase the medication convenience of the patient. In an example of the present invention, compared with the combination of copovidone with the commonly used excipients SDS, HPMC E5 and HPMCAS which have solubilization and/or dissolution enhancing effects in the art, when the dissolution-enhanced olaparib pharmaceutical composition of the present invention is prepared into tablets without addition of a filler and a disintegrant, olaparib can be smoothly dissolved (dissolution in 60 min is greater than 80%), but SDS, HPMC E5 and HPMCAS can not achieve efficient dissolution of olaparib from tablets (dissolution in 60 min is less than 80%).

The present inventors surprisingly discovered that, due to the combination of copovidone and the dissolution enhancer, the dissolution-enhanced olaparib composition of the present invention, which is compressed into tablets, has further increased exposure of olaparib, and superior in vivo absorption to the commercially available tablets and the preparations prepared by using co-povidone and mannitol in high amounts of excipients or the combination of povidone and the dissolution enhancer under the same amount of excipients, which is beneficial to further improve the utilization of the drug. In an example of the present invention, the dissolution-enhanced olaparib pharmaceutical composition of the present invention at the same dose of olaparib has an in vivo exposure in dog which is slightly higher than commercially available tablets (containing copovidone and mannitol) and the preparation prepared with copovidone and mannitol (with significant differences), and significantly higher than the preparation prepared with povidone and the cyclodextrin derivative.

The present inventors found that, for olaparib, the tablets prepared by using other water-soluble excipients commonly used in the art in combination with copovidone to enhance the dissolution of olaparib cannot achieve the effect of the combination of the dissolution enhancer and copovidone of the present invention. In an example of the present invention, when a solid dispersion is prepared by using copovidone alone and prepared into tablets without addition of mannitol in the formulation, the dissolution of olaparib in 60 min is less than 50%; the tablets prepared by using copovidone and SDS, copovidone and HPMC E5, or copovidone and HPMCAS (without addition of a disintegrant or a filler) have a dissolution (dissolution in 60 min is less than 70%) significantly slower than the preparation prepared by combination of copovidone and the dissolution enhancer of the present invention (dissolution in 60 min is greater than 80%); and the tablets prepared by enhancing dissolution with combination of povidone and the dissolution enhancer have also a dissolution significantly lower than that of the combination of copovidone and the dissolution enhancer of the present invention. It can be seen that the combination of copovidone and the water-soluble cyclodextrin derivative can achieve unexpectable effects.

The dissolution-enhanced olaparib medicament provided by the present invention can be a preparation suitable for transmucosal administration to a patient, that is, it can be administered to the mucosa for transmucosal absorption. Thus, suitable administration routes include administration by inhalation, as well as oral, intranasal and rectal administration. Oral administration is particularly preferred. A skilled person can select tablets, capsules or other preparation forms according to the administration route. However, other administration routes, such as parenteral administration, are not excluded. For example, the dissolution-enhanced olaparib medicament according to the present invention may be tablets, capsules, granules, pills, powders, etc., but is not limited thereto.

The dissolution-enhanced olaparib medicament of the present invention can be prepared by a person skilled in the art according to the dosage form using an appropriate method in the art. For example, in the case of tablets, a dissolution-enhanced olaparib composition can be prepared according to the formulaion, pulverized, added with other pharmaceutical excipients and mixed uniformly, compressed into tablets, and optionally, coated to give the tablets.

The dissolution-enhanced olaparib medicament according to the present invention may further include other pharmaceutical excipient, such as lubricants, glidants, coating agents, capsule materials, etc., but are not limited thereto. The other pharmaceutical excipient can be selected during preparation according to the needs of the dosage form.

The coating agent can improve taste and provide an exquisite appearance. If necessary, the coating agent may be an enteric coating agent. The coating agent usually contains a polymeric film-forming material, such as hydroxypropylmethyl cellulose, hydroxypropyl cellulose, and acrylate or methacrylate copolymer, but is not limited thereto. In addition to the film-forming polymer, the coating agent may also include a plasticizer such as polyethylene glycol, a surfactant such as Tweens, and an optional pigment such as titanium dioxide or iron oxide, but is not limited thereto. The coating agent may also contain talc as an anti-sticking agent.

In one embodiment, in general, the dissolution-enhanced olaparib medicament according to the present invention may include the following components:

| | |
|---|---|
| olaparib | 60 to 125, preferably 75 to 110 parts by weight |
| copovidone | 90 to 200, preferably 100 to 180 parts by weight |
| a dissolution enhancer | 20 to 100, preferably 25 to 90 parts by weight |
| a surfactant | 0 to 20, preferably 0 to 10, e.g., |
| | 0.1 to 10 parts by weight |
| other pharmaceutical | 0 to 80, preferably 2 to 30 parts by weight. |
| excipients | |

The other pharmaceutical excipients in the dissolution-enhanced olaparib medicament include glidants, lubricants, plasticizers, sweeteners, colorants, flavors, preservatives, coating agents, capsule materials, etc., but are not limited thereto. The description about these other pharmaceutical excipients is the same as the foregoing, and will not be repeated here.

The unit dosage form of the dissolution enhancing pharmaceutical preparation may contain 20 to 400 mg, preferably 50 to 300 mg of olaparib, but is not limited thereto. The patient can be an adult or a child, but other mammals are also expected to be treated, and the expected total amount to be taken per day is 100 to 1400 mg based on the active ingredient.

The dissolution-enhanced olaparib medicament of the present invention can be used to prevent or treat a tumor. Preferably, the tumor is selected from tumors with defective DNA repair functions, especially cancers associated with two or more BRCA gene mutations, such as ovarian cancer, gastric cancer, breast cancer, etc., and tumors associated with BRCA1 and BRCA2 gene mutations, but not limited thereto.

The olaparib as the active ingredient herein refers to olaparib free base or its pharmaceutically acceptable salts, such as hydrochloride, benzenesulfonate, sulfate, nitrate, camphorate and the like.

The term “surfactant” as used herein refers to a pharmaceutically acceptable surfactant.

The term “AUC” as used herein refers to the area under the concentration-time curve and has its conventional meaning, that is, the area under the plasma concentration-time curve for example from 0 to 24 hours. AUC has a unit of concentration multiplied by time. Once the test concentration-time point is determined, the AUC can be easily calculated, for example, by a computer program or by the trapezoidal method.

Compared with the existing olaparib solid dispersion preparation, the dissolution-enhanced olaparib composition provided by the present invention and the dissolution-enhanced olaparib medicament prepared therefrom have the following advantages:

1) Synergistic synergism: They have further enhanced solubilization ability for olaparib, and improve the supersaturated solubility and dissolution stability of olaparib compared with the cases of using copovidone or the dissolution enhancer alone. They have stable dissolution after a long-term storage with good stability, and can increase the exposure of olaparib in vivo;

2) Compound function, reducing the amount of excipients: it is not necessary to add a filler and/or a disintegrant in the formulation, the amount of excipients is reduced, the size of the preparation at the same specification is smaller than that of the commercially available tablets, which increases the medication convenience of the patient;

3) Increased dose: A higher single unit dose can be achieved to meet the needs of clinical high-dose medication.

### DESCRIPTION OF THE DRAWINGS

FIG. 1 shows in vitro dissolution curves (n=6) of various dissolution enhanced preparations prepared in Preparation Example 3, Comparative Example 1 and Comparative Example 2 of the present invention.

FIG. 2 shows in vitro dissolution curves (n=6) of various dissolution enhanced preparations prepared in Preparation Example 3 and Comparative Examples 3-7 of the present invention.

FIG. 3 shows in vitro dissolution curves (n=6) of various dissolution enhanced preparations prepared in Preparation Example 3, Comparative Example 3, Comparative Example 4 and Comparative Example 8 of the present invention at 0 month and 6 months under accelerated conditions.

FIG. 4 shows plasma concentration-time curves (n=3) of various dissolution enhanced preparations prepared according to Formulation 9 of Preparation Example 3, Comparative Example 1, and Comparative Example 3 of the present invention and commercially available tablets.

### BEST MODE

The following examples generally record the preparation method and/or characterization results of typical compositions of the present invention, and all percentages are by weight, unless otherwise specified. The following examples are specific illustrations of the present invention, and should not be considered as limiting the scope of the present invention. In the following embodiments, the processes and methods that are not described in detail are conventional methods known in the art.

In the present invention, the sources and trade names of the reagents and equipment used are indicated at the first appearance, and unless otherwise specified, the same reagents used thereafter are the same as those indicated for the first time. Conventional unindicated reagents are purchased from Sinopharm Chemical Reagent Co., Ltd. Among them, olaparib refers to the free base API, which is provided by Shanghai Biobond Pharmaceutical Co., Ltd.

Experimental animals: 12 beagle dogs, which are half male and half female and weigh 8-10 kg, were purchased from Beijing Marshall Biotechnology Co., Ltd. The test animals were adaptively reared in the test site of the Experimental Animal Center of Shanghai Institute of Materia Medica for 14 days before the test day.

### EXAMPLE

### Preparation Example 1

TABLE 1

| | Parts by weight | | | |
|---|---|---|---|---|
| Components | Formulation 1 | Formulation 2 | Formulation 3 | Formulation 4 |
| Olaparib | 84 | 105 | 81 | 96 |
| Copovidone | 162 | 145.5 | 144 | 120 |
| sulfobutyl-β-cyclodextrin | 45 | / | 66 | / |
| Hydroxypropyl-β-cyclodextrin | / | 43.5 | / | 75 |
| Colloidal silica | 3 | 4.5 | 6 | 3 |
| Sodium Stearyl Fumarate | / | 1.5 | 3 | / |
| PEG6000 | 6 | / | / | 6 |

Preparation method: Copovidone (PVP VA64, produced by BASF, Germany), the dissolution enhancer (sulfobutyl-β-cyclodextrin (produced by Cyclolab Ltd., Hungary), hydroxypropyl-β-cyclodextrin (produced by Roquette, France)), olaparib and colloidal silica (produced by Evonik Industries AG, Germany) were mixed and extruded with a twin-screw extruder (screw diameter 11 mm, Thermo Scientific company) to obtain a dissolution-enhanced olaparib composition.

The dissolution-enhanced olaparib composition prepared in this example was pulverized, added with other excipients according to the formulations in Table 1 and mixed uniformly, and compressed into tablets which contain 150 mg of olaparib per tablet by a single punch tablet machine. Among them, sodium stearyl fumarate is produced by JRS PHARMA GmbH & Co. KG, German, and PEG6000 is produced by Dow Chemical, USA.

### Preparation Example 2

TABLE 2

| | Parts by weight | | | |
|---|---|---|---|---|
| Components | Formulation 5 | Formulation 6 | Formulation 7 | Formulation 8 |
| Olaparib | 81 | 108 | 90 | 99 |
| Copovidone | 141 | 108 | 165 | 118.5 |
| sulfobutyl-β-cyclodextrin | 72 | / | 39 | / |
| Hydroxypropyl-β-cyclodextrin | / | 75 | / | 75 |
| Colloidal silica | 3 | 3 | 3 | 3 |
| Sodium Stearyl Fumarate | 3 | / | 3 | / |
| PEG6000 | / | 6 | / | 4.5 |

Preparation method: Copovidone (PVP VA64, produced by BASF, Germany), the dissolution enhancer (sulfobutyl-β-cyclodextrin, hydroxypropyl-β-cyclodextrin) and olaparib were dissolved in a solvent of methanol/acetone =1:4, evaporated to remove the solvent to obtain a dissolution-enhanced olaparib composition.

The dissolution-enhanced olaparib composition prepared in this example was pulverized, added with other excipients according to the formulations in Table 2 and mixed uniformly, and compressed into tablets which contain 150 mg of olaparib per tablet by a single punch tablet machine.

### Preparation Example 3

TABLE 3

| | Parts by weight | | | |
|---|---|---|---|---|
| Components | Formulation 9 | Formulation 10 | Formulation 11 | Formulation 12 |
| olaparib | 90 | 96 | 99 | 81 |
| Copovidone | 160.5 | 127.5 | 105 | 144 |
| Hydroxypropyl-β-cyclodextrin | 45 | 64.5 | 81 | 52.5 |
| Colloidal silica | 3 | 3 | 3 | 6 |
| labrasol | / | / | / | 9 |
| sodium lauryl sulfate | / | 6 | / | / |
| Span 20 | / | / | 9 | / |
| Sodium Stearyl Fumarate | 1.5 | / | 3 | 1.5 |
| glyceryl behenate | / | 3 | / | / |
| Opadry | / | / | / | 6 |

Among them, sodium lauryl sulfate is produced by BASF, Germany, glyceryl behenate and labrasol are produced by Gattefossé, France, and Span20 is produced by Nanjing Well Pharmaceutical co., LTD.

Preparation method: Copovidone, hydroxypropyl-β-cyclodextrin as the dissolution enhancer, olaparib, colloidal silica, labrasol, sodium lauryl sulfate and Span 20 were mixed and extruded with a twin-screw extruder to obtain a dissolution-enhanced olaparib composition.

The dissolution-enhanced olaparib composition prepared in this example was pulverized, added with other excipients (sodium stearyl fumarate and glyceryl behenate) according to the formulations in Table 3 and mixed well, and compressed into tablets which contain 150 mg of olaparib per tablet by a single punch tablet machine. Among them, the tablets obtained from Formulation 12 were taken, placed in a coating pan and film-coated with Opadry (produced by Shanghai Colorcon Coating Technology Co., Ltd.). Among them, Formulation 9 was compressed into tablets which contain 150 mg, 200 mg or 250 mg of olaparib per tablet with the tablet weight of 500 mg, 667 mg and 883 mg, respectively.

### Comparative Example 1

TABLE 4

| Components | Parts by weight |
|---|---|
| Olaparib | 75 |
| Copovidone | 172.5 |
| Colloidal silica | 5.5 |
| Mannitol | 44 |
| Sodium Stearyl Fumarate | 3 |

Preparation method: Copovidone, olaparib and colloidal silica were mixed according to the formulation in Table 4, and then the powdery mixture was extruded with a twin-screw extruder to obtain a dissolution-enhanced olaparib composition.

The obtained dissolution-enhanced olaparib composition was pulverized, added with other excipients according to the formulation in Table 4 and mixed uniformly, and compressed into tablets which contain 150 mg of olaparib per tablet by a single punch tablet machine.

### Comparative Example 2

TABLE 5

| Components | Parts by weight |
|---|---|
| Olaparib | 90 |
| Copovidone | 205 |
| Colloidal silica | 3 |
| Sodium Stearyl Fumarate | 1.5 |

Preparation method: Copovidone, olaparib and colloidal silica were mixed according to the formulation in Table 5, and then the powdery mixture was extruded with a twin-screw extruder to obtain a dissolution-enhanced olaparib composition.

The obtained dissolution-enhanced olaparib composition was pulverized, added with other excipients according to the formulation in Table 5 and mixed uniformly, and compressed into tablets which contain 150 mg of olaparib per tablet by a single punch tablet machine.

### Comparative Example 3

TABLE 6

| Components | Parts by weight |
|---|---|
| Olaparib | 90 |
| Povidone K25 | 160.5 |
| Hydroxypropyl-β-cyclodextrin | 45 |
| Colloidal silica | 3 |
| Sodium Stearyl Fumarate | 1.5 |

Preparation method: Povidone, olaparib and colloidal silica were dissolved in a solvent of methanol/acetone=1:4 according to the formulation in Table 6, and evaporated to remove the solvent to obtain a dissolution-enhanced olaparib composition.

The dissolution-enhanced olaparib composition was pulverized, added with other excipients according to the formulation in Table 6 and mixed uniformly, and compressed into tablets which contain 150 mg of olaparib per tablet by a single punch tablet machine.

### Comparative Example 4

TABLE 7

| Components | Parts by weight |
|---|---|
| Olaparib | 90 |
| Hydroxypropyl-β-cyclodextrin | 205.5 |
| Colloidal silica | 3 |
| Sodium Stearyl Fumarate | 1.5 |

Preparation method 1: Hydroxypropyl-β-cyclodextrin, olaparib and colloidal silica were mixed according to the formulation in Table 7, and then the powdery mixture was extruded with a twin-screw extruder. It was observed that the extrudate was whitish and opaque even when the temperature was higher than 230° C., and further increased temperature may deteriorate the stability of the drug and consume large energy, indicating that the formulation is not suitable for melt extrusion.

Preparation method 2: Hydroxypropyl-β-cyclodextrin and olaparib were dissolved in a solvent of methanol/acetone=1:4 according to the formulation in Table 7, and then evaporated to remove the solvent to obtain a dissolution-enhanced olaparib composition.

The dissolution-enhanced olaparib composition was pulverized, added with other excipients according to the formulation in Table 7 and mixed uniformly, and compressed into tablets which contain 150 mg of olaparib per tablet by a single punch tablet machine.

### Comparative Example 5

TABLE 8

| Components | Parts by weight |
|---|---|
| Olaparib | 90 |
| Copovidone | 190.5 |
| SDS | 15 |
| Colloidal silica | 3 |
| Sodium Stearyl Fumarate | 1.5 |

Preparation method: Copovidone, olaparib and sodium lauryl sulfate were dissolved in a solvent of methanol/acetone=1:4 according to the formulation in Table 8, and then evaporated to remove the solvent to obtain a dissolution-enhanced olaparib composition.

The dissolution-enhanced olaparib composition was pulverized, added with other excipients according to the formulation in Table 8 and mixed uniformly, and compressed into tablets which contain 150 mg of olaparib per tablet by a single punch tablet machine.

### Comparative Example 6

TABLE 9

| Components | Parts by weight |
|---|---|
| Olaparib | 90 |
| Copovidone | 160.5 |
| HPMC E5 | 45 |
| Colloidal silica | 3 |
| Sodium Stearyl Fumarate | 1.5 |

Preparation method: Copovidone, HPMC E5 (produced by Dow Chemical) and olaparib were dissolved in a solvent of methanol/acetone=1:4 according to the formulation in Table 9, and then evaporated to remove the solvent to obtain a dissolution-enhanced olaparib composition (it had to be prepared by the solvent method because the materials had significantly darkened color and degradation occurred when melt extrusion was performed at a temperature above 200° C.).

The dissolution-enhanced olaparib composition was pulverized, added with other excipients according to the formulation in Table 9 and mixed uniformly, and compressed into tablets which contain 150 mg of olaparib per tablet by a single punch tablet machine.

### Comparative Example 7

TABLE 10

| Components | Parts by weight |
|---|---|
| Olaparib | 90 |
| Hydroxypropyl-β-cyclodextrin | 160.5 |
| HPMCAS | 45 |
| Colloidal silica | 3 |
| Sodium Stearyl Fumarate | 1.5 |

Preparation method: Hydroxypropyl-β-cyclodextrin, HPMCAS (produced by Shin-Etsu Chemical Co., Ltd., Japan) and olaparib were dissolved in a solvent of methanol/dichloromethane=1:1 according to the formulation in Table 10, and then evaporated to remove the solvent to obtain a dissolution-enhanced olaparib composition.

The dissolution-enhanced olaparib composition was pulverized, added with other excipients according to the formulation in Table 10 and mixed uniformly, and compressed into tablets which contain 150 mg of olaparib per tablet by a single punch tablet machine.

### Comparative Example 8

TABLE 11

| Components | Parts by weight |
|---|---|
| Olaparib | 90 |
| Copovidone | 43.5 |
| Hydroxypropyl-β-cyclodextrin | 162 |
| Colloidal silica | 3 |
| Sodium Stearyl Fumarate | 1.5 |

Preparation method: Copovidone, olaparib and colloidal silica were mixed in a solvent of methanol/acetone=1:4 according to the formulation in Table 11, and then evaporated to remove the solvent to obtain a dissolution-enhanced olaparib composition.

The dissolution-enhanced olaparib composition was pulverized, added with other excipients according to the formulation in Table 11 and mixed uniformly, and compressed into tablets which contain 150 mg of olaparib per tablet by a single punch tablet machine.

### Experimental Example

### Experimental Example 1

### Solubility Test

The dissolution-enhanced olaparib compositions prepared in Preparation Examples 1-3 and Comparative Examples 1-8 and the olaparib API (free base) were taken in an amount equivalent to 10 mg of olaparib, dispersed in 5 ml of a pH 4.0 buffer (citric acid: 21 g/L: disodium hydrogen phosphate 71.63 g/L=61.45:38.55) as the solvent and shaken in a shaker at 100 rpm for 2 h at 37° C., and sampled. The samples were centrifugal filtered and measured by HPLC to determine the supersaturated solubility. The average value was obtained from 2 parallel runs. The results are shown in Table 12.

The operation conditions of the HPLC were:

Apparatus: A liquid chromatograph with a UV detector;

Chromatographic Waters Sunfire C18, 4.6 mm*50 mm

column:

Eluent: A: 0.1% TFA in water; B: 0.1% TFA in acetonitrile;

| | Time | A % | B % |
|---|---|---|---|
| Gradient program: | 0 | 75 | 25 |
| | 3.0 | 55 | 45 |
| | 3.5 | 0 | 100 |
| | 4.0 | 0 | 100 |
| | 7.0 | 75 | 25 |

Flow rate: 1 ml/min;

Temperature: 40° C.;

Wavelength: 276 nm;

Injection volume: 10 μl

TABLE 12
Test results of the supersaturated solubility of olaparib of the various formulations
Supersaturated solubility at 37° C. for 2 h (μg/ml)

| Olaparib API | Formulation 1 | Formulation 2 | Formulation 3 | Formulation 4 |
|---|---|---|---|---|
| 118.0 | 879.0 | 884.2 | 929.1 | 920.0 |
| Formulation 5 | Formulation 6 | Formulation 7 | Formulation 8 | Formulation 9 |
| 913.5 | 853.3 | 867.6 | 910.8 | 990.1 |
| Formulation | Formulation | Formulation | Comparative | Comparative |
| 10 | 11 | 12 | Example 1 | Example 2 |
| 952.5 | 933.3 | 1018.9 | 780.5 | 777.8 |
| Comparative | Comparative | Comparative | Comparative | Comparative |
| Example 3 | Example 4 | Example 5 | Example 6 | Example 7 |
| 825.5 | 1014.5 | 722.1 | 761.2 | 537.7 |

Comparative
Example 8
1001.9

It can be seen from the results that:

(1) Compared with the API, the combination of copovidone and the water-soluble cyclodextrin derivative significantly improves the supersaturated solubility of olaparib;

(2) Compared with copovidone alone, the combination of copovidone and the water-soluble cyclodextrin derivative further improves the supersaturated solubility of olaparib (>850 μg/ml vs about 780 μg/ml);

(3) Compared with the combination of copovidone with SDS, a commonly used excipient with dissolution enhancing effect, or with HPMC E5 or HPMCAS, the commonly used excipients with solubilizing effect, the combination of copovidone and the water-soluble cyclodextrin derivative as the dissolution enhancer can further improve the supersaturated solubility of olaparib;

(4) Compared with the water-soluble cyclodextrin derivative alone, the combination of copovidone and the water-soluble cyclodextrin derivative has the equivalent effect on enhancing the dissolution of olaparib, but with better stability under the same dosage ratio of olaparib and excipients;

(5) Compared with the combination of povidone and the water-soluble cyclodextrin derivative, the combination of copovidone and the water-soluble cyclodextrin derivative has a better effect on improving the supersaturated solubility of olaparib (990.1 μg/ml vs 825.5 μg/ml).

### Experimental Example 2

### Dissolution Test

The dissolution behaviors of the preparations prepared in Preparation Examples 1-3 and Comparative Examples 1-7 were measured according to the following dissolution test method.

The dissolution of the obtained tablets (150 mg specification) was measured on the apparatus of the third method of the dissolution test method (Chinese Pharmacopoeia 2015, Volume IV, General Chapters, 0931). The operation was performed according to the method at 37° C. in 250 ml of a pH 4.0 buffer (citric acid 21 g/L: disodium hydrogen phosphate 71.63 g/L=61.45:38.55) as the release medium on a dissolution apparatus at 50 rpm. 2 ml of a solution was taken after 15, 30, 60, 90, 120, 180 min respectively, and centrifuged. The supernatant was diluted by one time with an acetonitrile-water (1:1) solution as the solvent to be used as the test solution, and the dissolution was determined by HPLC, and 6 runs were operated in parallel.

The operation conditions of the HPLC were:

Apparatus: A liquid chromatograph with a UV detector;

Chromatographic Waters Sunfire C18, 4.6 mm*50 mm

column:

Eluent: A: 0.1% TFA in water; B: 0.1% TFA in acetonitrile;

| | Time | A % | B % |
|---|---|---|---|
| Gradient program: | 0 | 75 | 25 |
| | 3.0 | 55 | 45 |
| | 3.5 | 0 | 100 |
| | 4.0 | 0 | 100 |
| | 7.0 | 75 | 25 |

Flow rate: 1 ml/min;

Temperature: 40° C.;

Wavelength: 276 nm;

Injection volume: 10 μl

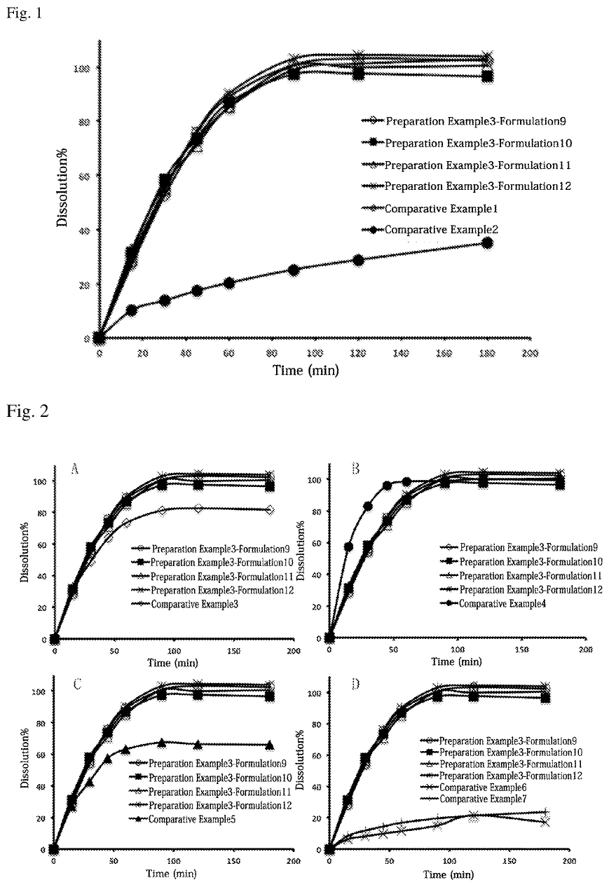

The results of dissolution determination are shown in FIGS. 1-2. The results show that:

(1) The dissolution in 60 min of olaparib in formulations 9-12 was greater than 80%, which is consistent with that of the formulation of Comparative Example 1 containing 14.7% of mannitol, indicating that olaparib in the tablets of the present invention can be effectively dissolved without addition of a filler and a disintegrant;

(2) For the solid dispersion prepared by using copovidone alone, olaparib cannot be effectively dissolved after the solid dispersion is prepared into tablets, if mannitol was not added into the formulation;

(3) The olaparib tablets (without addition of a disintegrant or a filler) prepared by using combination of povidone and the water-soluble cyclodextrin derivative as the dissolution enhancer, copovidone and SDS, copovidone and HPMC E5 or copovidone and HPMCAS, have significantly slower dissolution than the preparation prepared by using combination of copovidone and the water-soluble cyclodextrin derivative as the dissolution enhancer;

(4) The olaparib tablets prepared by using the water-soluble cyclodextrin derivative has a faster dissolution of olaparib in 60 min and a consistent dissolution of olaparib in 90 min compared with the preparation prepared by using combination of copovidone and the water-soluble cyclodextrin derivative.

### Experimental Example 3

### Stability Study

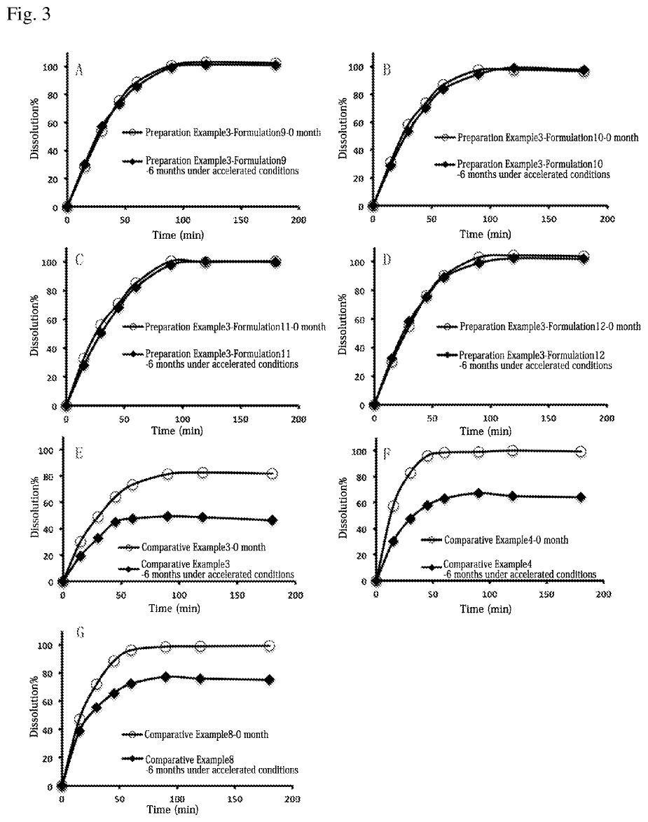

The solid olaparib preparations prepared in Preparation Example 3, Comparative Example 3, Comparative Example 4, and Comparative Example 8 of the present invention were placed under accelerated conditions at 40±2° C. and 75%±5% RH for 6 months, and the dissolution behavior (n=6) of the drug was determined following the conditions described in Experimental Example 1, and the dissolution curves were plotted (see FIG. 3).

It can be seen from the results that the solid olaparib preparations prepared in Preparation Example 3 of the present invention had significantly improved dissolution stability of olaparib, compared with the solid olaparib preparation of Comparative Example 3 prepared by using combination of povidone and the water-soluble cyclodextrin derivative, the solid olaparib preparation of Comparative Example 4 prepared by using the water-soluble cyclodextrin derivative alone, and the solid olaparib preparation of Comparative Example 8 prepared by using combination of copovidone and the water-soluble cyclodextrin derivative in a ratio that is not preferred in the present invention.

### Experimental Example 4

### Pharmacokinetic Studies

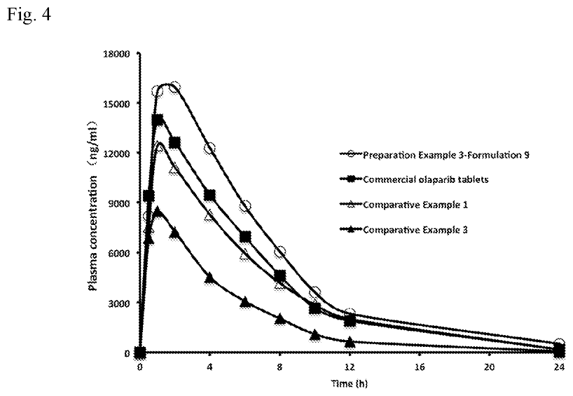

The preparation of Formulation 9 of Preparation Example 3, the commercially available tablets (150 mg specification, Lynparza®, produced by AbbVie, Germany), and the preparations of Comparative Examples 1 and 3 (150 mg specification) were respectively administered to fasted beagle dogs (n=3) with 50 mL of water separately. Blood was collected at 0 h, 0.5 h, 1 h, 2 h, 4 h, 6 h, 8 h, 10 h, 12 h, and 24 h after administration. The blood sample was centrifuged at 4° C. at 4000 rpm for 10 min. The upper plasma was taken and used for plasma concentration detection by LC-MS. The results are shown in FIG. 4 and Table 13.

It can be seen from the plasma concentration-time curves of FIG. 4 and the results of Table 13 that, compared to the commercially available tablets, Comparative Example 1 (copovidone and mannitol) and Comparative Example 3 (povidone and the water-soluble cyclodextrin derivative), the preparation of the formulation 9 of Preparation Example 3 had a significantly improved AUC (the formulation 9 had an AUC which is higher by 25.9% than that of commercially available tablets, by 38.4% than that of Comparative Example 1, and by 156.7% than that of Comparative Example 3), the preparation of the formulation 9 does not need to add additional mannitol to promote the dissolution of olaparib, and the amount of excipients can be reduced by more than 100 mg compared with the commercially available tablets and Comparative Example 1, and the preparation of the formulations 9 is easy for the patient to swallow, and meanwhile, it provides a larger dose space for increasing the drug dose and exerting the best effect.

TABLE 13
Pharmacokinetic parameters in dogs after administration of various preparations (n =3)

| | | | Relative |
|---|---|---|---|
| | Cmax | AUC_{(0-24 h)} | bioavailability |
| Preparation | (μg/ml) | (μg:h/ml) | (%) |
| Formulation 9, | 15.93 ± 1.87 | 120.67 ± 8.65 | 125.9% |
| Preparation Example 3 | | | |
| Commercial olaparib | 14.35 ± 1.22 | 95.83 ± 6.73* | 100.0% |
| tablets | | | |
| Comparative Example 1 | 14.00 ± 1.20 | 87.22 ± 6.04* | 91.0% |
| Comparative Example 3 | 8.67 ± 1.32* | 47.0 ± 3.91* | 49.0% |

Remarks:
*Compared with the Cmax and AUC of the formulation 9 of Preparation Example 3, P < 0.05 after statistically calculation.

1. A dissolution-enhanced olaparib composition, comprising: olaparib; copovidone and a dissolution enhancer;
wherein, in the dissolution-enhanced olaparib composition, based on 100 parts by weight of olaparib, the copovidone is 100 or more and less than 200, and the dissolution enhancer is 20 to 150.

2. The dissolution-enhanced olaparib composition of claim 1, wherein the dissolution enhancer is selected from water-soluble cyclodextrin derivatives.

3. The dissolution-enhanced olaparib composition of claim 1, further comprising other pharmaceutical excipients selected from the group consisting of surfactants, glidants, lubricants, and plasticizers.

4. A method for preparing the dissolution-enhanced olaparib composition of claim 1, comprising mixing olaparib with copovidone, the dissolution enhancer and the optional other pharmaceutical excipients uniformly to obtain a uniform dispersion.

5. (canceled)

6. (canceled)

7. A dissolution-enhanced olaparib medicament, comprising the dissolution-enhanced olaparib composition of claim 1.

8. The dissolution-enhanced olaparib medicament of claim 7, further comprising other pharmaceutical excipient, wherein the other pharmaceutical excipient is one or more selected from the group consisting of lubricants, glidants, and coating agents.

9. The dissolution-enhanced olaparib medicament of claim 7, wherein the medicament is a preparation suitable for transmucosal administration to a patient.

10. A method for prevention or treatment of a tumor, comprising administering a subject in need thereof the dissolution-enhanced olaparib medicament of claim 7.

11. The dissolution-enhanced olaparib composition of claim 1, wherein, in the dissolution-enhanced olaparib composition, based on 100 parts by weight of olaparib, the copovidone is 150 to 195 parts by weight, and the dissolution enhancer is 25 to 120 parts by weight.

12. The dissolution-enhanced olaparib composition of claim 1, wherein the dissolution enhancer is one or a combination of two or more selected from methyl-β-cyclodextrin, hydroxypropyl-β-cyclodextrin, sulfobutyl-β-cyclodextrin, and hydroxypropyl-γ-cyclodextrin.

13. The dissolution-enhanced olaparib composition of claim 1, wherein the dissolution enhancer is hydroxypropyl-β-cyclodextrin, sulfobutyl-β-cyclodextrin, or a combination thereof.

14. The dissolution-enhanced olaparib composition of claim 3, wherein,
the surfactant is one or more selected from sodium lauryl sulfate, docusate sodium, cetrimide, benzethonium chloride, cetylpyridinium chloride, lauric acid, polyoxyethylene alkyl ether, sorbitan fatty acid ester, polyoxyethylene sorbitan fatty acid ester, polyoxyethylene castor oil derivative, polyoxyl 40 stearate, octyl/decyl mono and diglycerides, polyoxyethylene stearate and poloxamer,
the glidant is one or more selected from colloidal silica, animal or vegetable fats, and waxes, and
the lubricant is one or more selected from polyethylene glycol, magnesium stearate, calcium stearate, sodium stearyl fumarate, glyceryl mono/dibehenate, polyethylene glycol glyceryl behenate and glyceryl distearate.

15. The dissolution-enhanced olaparib composition of claim 3, wherein, based on 100 parts by weight of olaparib,
the surfactant is 0 to 20 parts by weight,
the glidant is 0 to 15 parts by weight, and
the lubricant is 0 to 15 parts by weight.

16. The dissolution-enhanced olaparib composition of claim 3, wherein, based on 100 parts by weight of Olaparib,
the surfactant is 0-10 parts by weight,
the glidant is 0 to 10 parts by weight, and
the lubricant is 0-10 parts by weight.

17. The dissolution-enhanced olaparib medicament of claim 9, wherein the preparation is a tablet.

18. The method of claim 10, wherein the tumor is selected from tumors with defective DNA repair function.

19. The method of claim 10, wherein the tumor is selected from cancers associated with two or more BRCA gene mutation.

20. The method of claim 10, wherein the tumor is selected from ovarian cancer, gastric cancer, breast cancer, and tumors associated with BRCA1 and BRCA2 gene mutations.
